(19) United States
(12) Patent Application Publication
(10) Pub. No.: US 2024/0180825 A1
(43) Pub. Date: Jun. 6, 2024

(54) Polymer Liposome Hydrogel

(71) Applicant: THE BOARD OF TRUSTEES OF THE LELAND STANFORD JUNIOR UNIVERSITY, Stanford, CA (US)

(72) Inventors: Santiago Correa, Palo Alto, CA (US); John Klich, Freehold, NJ (US); Eric Andrew Appel, Stanford, CA (US)

(21) Appl. No.: 18/285,315

(22) PCT Filed: Apr. 19, 2022
(86) PCT No.: PCT/US2022/025315

§ 371 Date: Oct. 2, 2023

Related U.S. Application Data
(60) Provisional application No. 63/177,373, filed on Apr. 20, 2021.

(51) Int. Cl. A61K 9/06; A61K 9/00; A61K 9/127; A61K 47/38

(52) U.S. Cl. CPC A61K 9/06; A61K 9/0019; A61K 9/127; A61K 47/38

(57) ABSTRACT

An injectable hydrogel network is provided distinguishing a non-covalently cross-linked hydrogel network with polymers functionalized with hydrophobic fatty pendant groups hydrophobically cross-linked with liposomal nanoparticles in which the liposomal nanoparticles are the cross-linkers. As the hydrogel is injectable, it is easily administered under the skin and does not require invasive surgical implantation. After injection the hydrogel rapidly self-heals to form a robust solid-like depot that can persist in the body over relevant timescales. The material does not cause a negative immune response like many other materials do. The hydrogel can gradually degrade over time in the body and therefore would not require surgical removal. Different embodiments modifying the surface chemistry of the liposomes give this hydrogel the ability to release therapeutic cargo, such as proteins, over controlled programmable timescales regardless of cargo size because the material can interact with the cargo through electrostatic or affinity interactions.

### FIELD OF THE INVENTION

This invention relates to injectable hydrogel networks. In particular, the invention relates to the field of drug/cellular delivery and tissue regeneration.

### BACKGROUND OF THE INVENTION

Protein drugs are essential tools for engineering biological systems to improve human health. Challenging biomedical applications, such as tissue regeneration and wound healing, often rely on diverse growth factors and cytokines. Likewise, immunotherapy relies heavily on antibodies, engineered proteins, cytokines, and chemokines to activate or dampen the immune system (e.g., for cancer or autoimmune disorders, respectively). While tissue regeneration, wound healing, and immuno-engineering pursue different biological outcomes, they share a similar challenge to manipulate complex biological networks. The capacity to successfully rewire these networks rests on the ability to perturb multiple dynamic signaling pathways at specific times and within specific tissues (e.g., diseased tissues or specific lymphoid organs) to achieve desired therapeutic outcomes. Indeed, disruption of these networks outside of the appropriate time frame or outside of the target tissues can lead to serious side effects. Taken together, technology that can precisely coordinate the release kinetics of diverse protein drugs in specific locations is critical for the controlled modulation of biological systems.

Nanomedicine and injectable hydrogel technologies seek to provide precise spatiotemporal control over drug delivery. In terms of spatial control, nanoparticle drug carriers seek to preferentially accumulate in target tissues after systemic administration, but besides filtration organs like the liver and spleen, their ability to target to specific tissues is limited. In terms of scheduled multi-drug release, nanomedicine has been successful in staging the release of different classes of drugs (e.g., hydrophilic versus hydrophobic small molecules, nucleic acids, and proteins), but staged delivery of proteins remains a challenge. Meanwhile, the emergence of injectable hydrogels has provided a significant improvement for minimally invasive localized therapy, but scheduled multi-drug release from these systems is often limited to strategies that leverage size-governed (e.g., smaller drugs first) or solubility-governed (e.g., most soluble drugs first) release mechanisms. Overall, there remains no minimally invasive delivery technology that can tune the relative release rates of multiple protein drugs in vivo.

The present invention addresses this technology gap.

### SUMMARY OF THE INVENTION

The present invention provides an injectable hydrogel network. The network is a non-covalently cross-linked hydrogel network with polymers functionalized with hydrophobic fatty pendant groups hydrophobically cross-linked with liposomal nanoparticles in which the liposomal nanoparticles are the cross-linkers. In one embodiment, the polymers are functionalized with hydrophobic fatty pendant groups are dodecyl-modified hydroxypropyl methyl cellulose. In a further embodiment, the liposomal nanoparticles have affinity motifs capable of carrying and delivery of cargo. In yet a further embodiment, the liposomal nanoparticles have phospholipids capable of carrying and delivery of cargo. In yet a further embodiment, the hydrophobic fatty pendant groups are defined as covalently attached functional groups bearing hydrophobic chemistries. In still a further embodiment, the attached functional groups are alkyl, alkenyl, alkynl, or aryl functional groups.

The hydrogel material is injectable. Therefore, it is easily administered under the skin and does not require invasive surgical implantation as many other hydrogel materials do. After injection the hydrogel rapidly self-heals to form a robust solid-like depot that can persist in the body over relevant timescales. The material does not cause a negative immune response like many other materials do. Compared to other hydrogels, it can be an advantage that this material gradually degrades over time in the body and does not require surgical removal. The material is composed of liposomes, and liposomes can be easily formed with varying surface chemistries. The different embodiments modifying the surface chemistry of the liposomes give this material the ability to release therapeutic cargo, such as proteins, over controlled programmable timescales regardless of cargo size because the material can interact with the cargo through electrostatic or affinity interactions. This is a great advantage compared to most other hydrogel materials that only release drug cargo based of the cargo size through passive diffusion mechanisms at much faster timescales.

### BRIEF DESCRIPTION OF THE DRAWINGS

For grey-scale interpretation of the drawings, the reader is referred to the priority document.

FIG. 1 shows according to an exemplary embodiment of the invention the formation of liposomal hydrogels through mixing liposomal nanoparticles with polymers functionalized with hydrophobic fatty pendant groups.

FIG. 2 shows according to an exemplary embodiment of the invention liposomal hydrogels formed from functionalized liposomes to regulate release rate of cargo.

FIG. 3 shows according to an exemplary embodiment of the invention a depiction of the hydrogel network composed of dodecyl-modified hydroxypropyl methyl cellulose polymer and liposomal nanoparticles. Supramolecular hydrophobic interactions between the polymer and the nanoparticle lead to crosslinking of the network and formation of a hydrogel.

FIG. 4 shows according to an exemplary embodiment of the invention a demonstration of dynamic rheological properties of polymer-liposome hydrogels. Properties are tunable by altering the concentration (weight percent) of liposomes in the formulation.

FIG. 5 shows according to an exemplary embodiment of the invention a summary of key rheological parameters varying with liposomal content of hydrogels. G′, G″, and tan(delta) values taken for 10 rad/s.

FIG. 6 shows according to an exemplary embodiment of the invention dynamic rheological properties of polymer-liposome hydrogels are further tunable by altering the size of liposomes within the formulation.

FIG. 7 shows according to an exemplary embodiment of the invention polymer-liposome hydrogels are injectable through fine needles for minimally invasive clinical usage.

FIG. 8 shows according to an exemplary embodiment of the invention polymer-liposome hydrogels possess extensional rheological attributes that are tunable by the size of the liposomes implemented in the formulation.

FIG. 9 shows according to an exemplary embodiment of the invention polymer-liposomes are biocompatible. These hydrogels exhibit controlled erosion in vivo in C57BL/6 mice and do not provoke any signs of toxicity or inflammation. Scale bar is 25 mm.

FIG. 10 shows according to an exemplary embodiment of the invention surface chemistry of liposomes can be engineered to provide additional functionality to polymer-liposome hydrogels. Demonstrated is the inclusion of NTA-modified phospholipids into the liposomal component of the hydrogels. The resulting hydrogel is able to slow the release of a model his-tagged protein (GFP) in vitro. Data are % of total mass released based on amount loaded into hydrogels.

FIG. 11 shows according to an exemplary embodiment of the invention histological analysis of explanted liposomal hydrogels using H&E or trichrome staining indicate minimal inflammatory response to the material following a week of implantation in immunocompetent mice. These indicate an overall lack of a foreign body response which indicates the material is nonimmunogenic and suitable for long term and sustained drug delivery applications.

FIG. 12 shows according to an exemplary embodiment of the invention further data demonstrating safety and biocompatibility in immunocompetent mice. Caliper measurements of hydrogel depot size over time indicates steady erosion of the material in vivo. Stable body weights of hydrogel treated mice are consistent with body weights of untreated mice. Blood chemistry panel on serum collected 7 days after hydrogel injection. Liver function was tracked by measuring AST/ALT ratio and ALP levels. Kidney function was tracked by measuring serum BUN and creatinine levels. All blood chemistry measures were consistent between hydrogel treated and untreated control mice.

FIG. 13 shows according to an exemplary embodiment of the invention liposomal hydrogels enable synchronized and sustained co-delivery of IgG antibody and IL-12 cytokine cargo in vivo, despite substantial size difference. Liposomal hydrogel mesh size is able to significantly slow release of bulky IgG antibodies, while anionic liposomes regulate release of small IL-12 cytokines via electrostatic interactions. Representative IVIS images of Alexa Fluor 594 labeled IgG and Alexa Fluor 700 labeled IL-12 release over time. Quantified release curves of IgG and IL-12 following local subcutaneous bolus injection or subcutaneous injection of liposomal hydrogel.

FIG. 14 shows according to an exemplary embodiment of the invention IL-12 release in vivo is tuned by liposome surface chemistry, whereas IgG which lacks the affinity motif is unaffected by surface chemistry change. These data indicate this technology is capable of orthogonally modifying the release properties of co-encapsulated proteins. Mice were injected with liposomal hydrogels containing fluorescently labeled IgG and his-tagged IL-12. Area under the drug-time curve was calculated for in vivo release data for IgG and IL-12 for each administration method.

### DETAILED DESCRIPTION

Controlled spatiotemporal manipulation of biological processes are currently not readily available. Numerous diseases and injuries would benefit from controlling the location and duration of action of bioactive compounds (e.g. drugs, therapeutic cells, scaffolds). Injectable, macroscopic hydrogels can address this challenge, because they permit minimally invasive introduction of a drug vehicle precisely at the tissue of interest.

This invention describes a supramolecular hydrogel composed of polymers and liposomal/lipid-based nanoparticles. Embodiments of the invention are injectable, and the material properties are tunable by altering the concentration of the base components (polymer and liposome) as well as through modification of the liposome size and nanoscale architecture. In general, the invention can be described by polysaccharides bearing hydrophobic modifications and lipid-based nanoparticles. Examples of each are: polysaccharides including hydroxypropyl methyl cellulose, other cellulose derivatives, hyaluronic acid, chitosan, dextran, or xanthan modified with hydrophobic pendant groups that may include alkyl, alkenyl, alkynl, and aryl functional groups. Lipid-based nanoparticles include liposomes and solid lipid nanoparticles.

The inclusion of liposomes into the hydrogel network provides highly stable retention of the nanoparticles in the hydrogel, and provides access to the numerous biomedical capabilities of liposomes. These include the encapsulation of hydrophobic and hydrophilic drugs, a high level of biocompatibility, ease in engineering nanoscale architecture, capacity to mimic cells (e.g. artificial antigen presenting cells), and engineered surface chemistries. As a result, embodiments of this invention would have broad applications across medical conditions requiring local and sustained introduction of bioactive compounds.

Embodiments of the invention have broad applications for local drug delivery and tissue regeneration. In particular, they may be relevant for local immunomodulation (e.g. for cancer immunotherapy or infectious disease vaccines) and for cellular therapies (e.g. CAR T cell, stem cell, autologous dendritic cell delivery). Embodiments of the invention could also be useful for cell culture in vitro, in particular for 3D cell culture and ex vivo stimulation/expansion of cells.

The incorporation of liposomes into the molecular network of the hydrogel is a major advantage of the embodiments. Prior art looking to employ liposome or lipid nanoparticle technology intro hydrogels use physical entrapment, which negatively impacts retention of these nanoparticles. Moreover, because embodiments of the invention are assembled through dynamic supramolecular interactions between liposomes and the hydroxypropyl methyl cellulose (HPMC) polymer, it is injectable and allows facile and minimally invasive implementation in clinical settings. The ability to engineer the surface chemistry of the liposomal nanoparticle component allows the incorporation of new functions including cell-binding and affinity interactions with therapeutic cargo. The formation of the hydrogel network is robust to the liposome formulation, making it compatible with a wide range of formulations.

In one embodiment the invention is an injectable hydrogel network. This network is a non-covalently cross-linked hydrogel network with polymers functionalized with hydrophobic fatty pendant groups hydrophobically cross-linked with liposomal nanoparticles in which the liposomal nanoparticles are the cross-linkers. In this embodiment, polymers functionalized with hydrophobic fatty pendant groups are dodecyl-modified hydroxypropyl methyl cellulose. In one variation, the liposomal nanoparticles may further have affinity motifs capable of carrying and delivery of cargo. In another variation, the liposomal nanoparticles may further have phospholipids capable of carrying and delivery of cargo. Hydrophobic fatty pendant groups are defined as covalently attached functional groups bearing hydrophobic chemistries. The attached functional groups are alkyl, alkenyl, alkynl, or aryl functional groups.

Embodiments of the invention exclude polymers which are modified with cholesterol, which is a moiety that is unable to mediate sufficiently strong hydrophobic interactions with liposomes to yield a robust hydrogel that exhibits solid-like properties (G′ greater than G″) at low frequencies (e.g. 10⁻¹ to 10 rads/s). The poor overall mechanical properties, and inability to tune those properties, limit the functionality of the hydrogel for clinical applications.

Embodiments of the invention exclude polymers which mediate interactions with liposomes via cholesterol-modified DNA-based cross-linkers. Similar to cholesterol-modified polymers, these gels exhibit poor mechanical properties that cannot be tuned. Moreover, the abundance of nucleases within the body, which can readily degrade the DNA linkages and drive dissolution of the hydrogel and premature release of liposomes, limits the clinical utility of such hydrogels.

Embodiments of the invention also exclude polymers modified with functional groups that mediate dynamic covalent crosslinks, such as Schiff base reactions involving aldehydes and amines. The usage of aldehydes introduces biocompatibility concerns for these systems, as these functional groups can readily react with free amines (e.g. lysine residues) in the body, leading to unanticipated toxicities. Moreover, lipid nanoparticle formulations in such systems are constrained to those that include amine-bearing phospholipids, which limits the functionalities provided by liposomes.

### Hydrogel Assembly

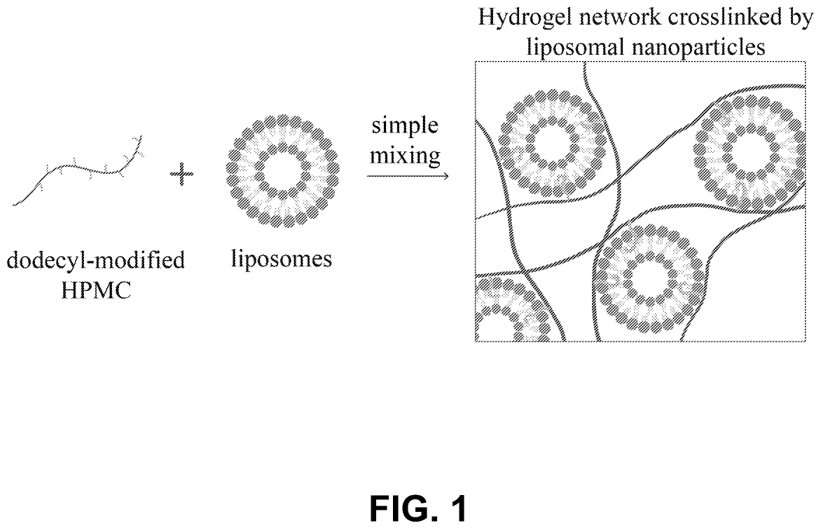

FIG. 1 shows the formation of liposomal hydrogels through mixing liposomal nanoparticles with polymers functionalized with hydrophobic fatty pendant groups. Here, dodecyl-modified hydroxypropyl methyl cellulose is combined with liposomes. Liposome were formed form a variety of zwitterionic (phosphocholine headgroups) and anionic phospholipids (phospho-(1′-rac-glycerol) headgroups) and cholesterol, generally combined at a molar ratio of 9:2:1, respectively. Lipids could be prepared in a range of ratios, such as a molar ratios of 9:2:0, 9:2:1, 9:2:4, or 9:0:1, with each ratio corresponding to the phophocholine, phospho-(1′-rac-glycerol), and cholesterol components, respectively. Hydrophobic interactions between the dodecyl pendant chains and the lipid bilayer yield a highly cross-linked network, wherein the liposomes themselves act as the cross-linkers. These interactions are dynamic and reversible, which allows the material to exhibit shear-thinning and self-healing capabilities necessary to be injectable.

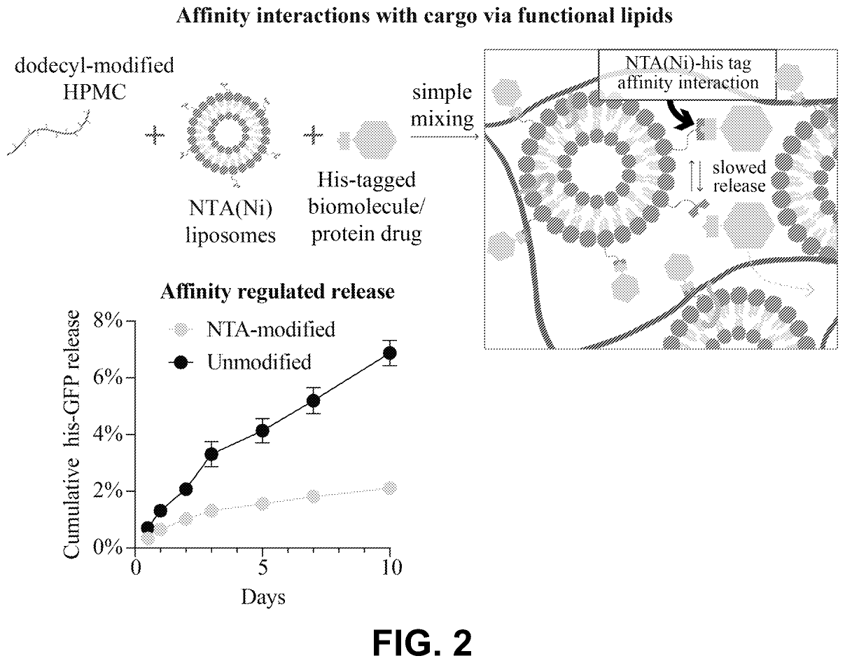

FIG. 2 shows liposomal hydrogels formed from functionalized liposomes to regulate release rate of cargo. The liposome component can be engineered to exhibit affinity motifs. In an exemplary embodiment, the inventors demonstrate that liposomes that include NTA(Ni) functionalized phospholipids exhibit specific interactions with histidine-tagged biomolecules. When all components (polymer, liposome, cargo) are combined to form a gel, the NTA(Ni) modified formulation significantly slows down the release rate of histidine-tagged green fluorescent protein.

### Method of Making

### Preparation of Dodecyl-Modified Hydroxypropylmethyl Cellulose

Hypromellose (1 g) was dissolved in anhydrous NMP (40 mL) and heated to 80 degrees Celsius in a PEG bath. Dodecyl isocyanate (125 μL) was diluted in anhydrous NMP (5 mL) and added dropwise to the Hypromellose solution while rapidly stirring. HUNIGs catalyst (10 drops) was added dropwise to the reaction solution while rapidly stirring. The heat bath was turned off, and the reaction was allowed to continue overnight while stirring. The polymer was precipitated in acetone (600 mL) and then dissolved in Millipore water (˜40 mL) prior to being dialyzed (3.5 kDa MWCO) for 4 days. Pure HPMC-C12 was then lyophilized and dissolved in PBS to produce a 6% w/v solution, which was stored at 4 degrees Celsius until used.

### Preparation of Liposomes

Liposomes were prepared from a variety of phospholipid formulations that include 1,2-distearoyl-sn-glycero-3-phosphocholine (DSPC), 1,2-distearoyl-sn-glycero-3-phospho-(1′-rac-glycerol) (DSPG), 1,2-dimyristoyl-sn-glycero-3-phosphocholine (DMPC), 1,2-dimyristoyl-sn-glycero-3-phospho-(1′-rac-glycerol) (DMPG), 1,2-dioleoyl-sn-glycero-3-phosphocholine (DOPC), 1,2-dioleoyl-sn-glycero-3-phospho-(1′-rac-glycerol) (DOPG), 1,2-dioleoyl-sn-glycero-3-[(N-(5-amino-1-carboxypentyl)iminodiacetic acid)succinyl] (nickel salt) (DGS-NTA(Ni)), and cholesterol. For synthesis, liposome phospholipid ratios were held at 9:2:1 molar ratio of a phosphocholine (e.g., DSPC, DMPC, DOPC), phospho-(1′-rac-glycerol) (e.g. DSPG, DMPG, DOPG), and cholesterol, respectively. Phospholipid mixtures were combined in chloroform in round-bottom flasks, and lipid films were formed using a rotary evaporator. The films were rehydrated using phosphate buffered saline under agitation (rotation or vortexing) at 40 degrees Celsius to create a solution of large, crude, multi-lamellar liposomes. The crude solution was then extruded through polycarbonate membranes of decreasing pore size (400 nm to 200 nm to 100 nm to 50 nm) using a handheld extrusion device (Avanti Mini Extruder) until a uni-lamellar species of uniform size was generated. For the inclusion of functional phospholipids (e.g., DGS-NTA(Ni)) the functional lipid is incorporated into the initial lipid mixture in chloroform at the desired mole fraction, and then synthesis proceeds as described above. Alternatively, the functional lipid can be post-inserted into the fully formed uni-lamellar liposomes as follows. First, the amount of functional lipid to be incorporated is prepared in chloroform in glassware, and treated to rotary evaporation to form a dry thin film. The film is then re-hydrated using the pure uni-lamellar liposome solution at 40 degrees Celsius for a period of 4 to 24 hours. Concentration of liposome solutions were carried out using Amicon centrifugal filter units.

### Preparation of Liposome Hydrogel

Liposome solutions were prepared in phosphate buffered saline (PBS) to a concentration of 15 weight percent lipid. Liposome solutions were diluted as necessary in PBS based on the desired final weight percent of lipid in the final hydrogel. Liposome solution was then added directly to the aqueous 6 weight percent solution of HPMC-C12, and mixed with a spatula until gelation occurs. The final composition of the gel formulations reported here were all 2 weight percent HPMC-C12 and varied in liposome content (1, 4, and 10 weight percent lipids). For sterile preparation, the liposome and HPMC solutions are drawn into two sterile syringes, one for each solution. The two syringes are connected using an autoclaved elbow luer lock connector, and the solution are vigorously mixed together by alternatively pressing the syringe plungers. Gelation is confirmed through dynamic rheological characterization.

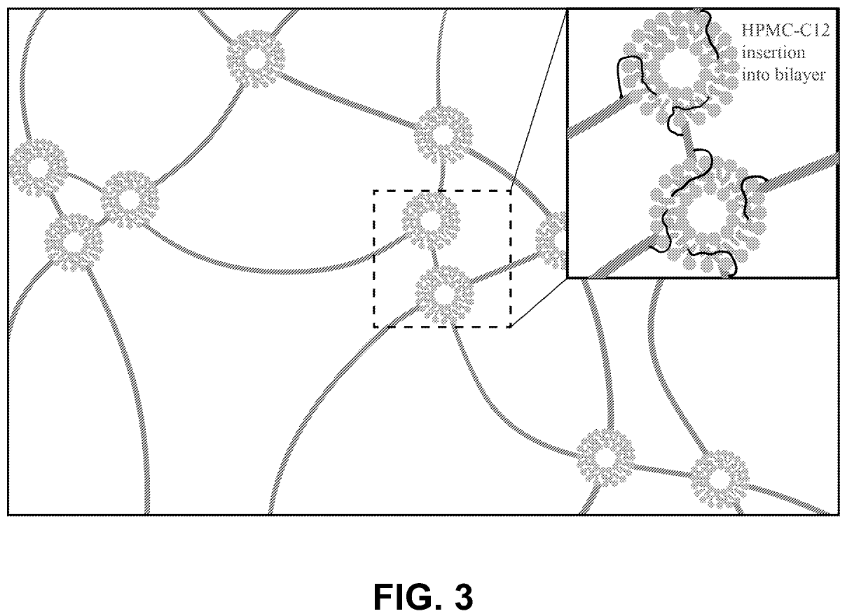

FIG. 3 shows a depiction of the hydrogel network composed of dodecyl-modified hydroxypropyl methyl cellulose polymer and liposomal nanoparticles. Supramolecular hydrophobic interactions between the polymer and the nanoparticle lead to crosslinking of the network and formation of a hydrogel.

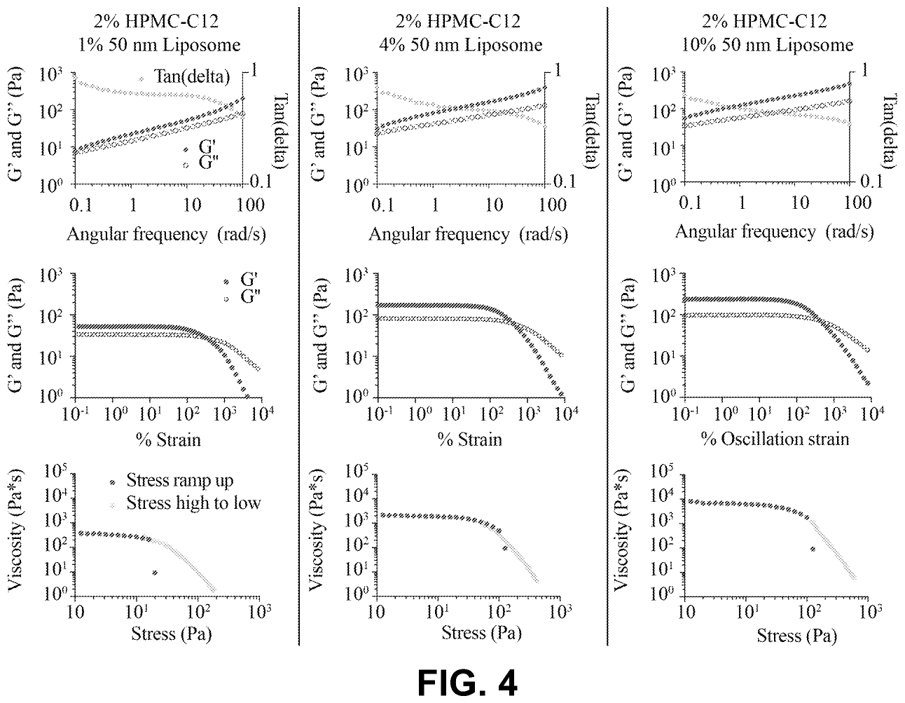

FIG. 4 shows a demonstration of dynamic rheological properties of polymer-liposome hydrogels.

Properties are tunable by altering the concentration (weight percent) of liposomes in the formulation.

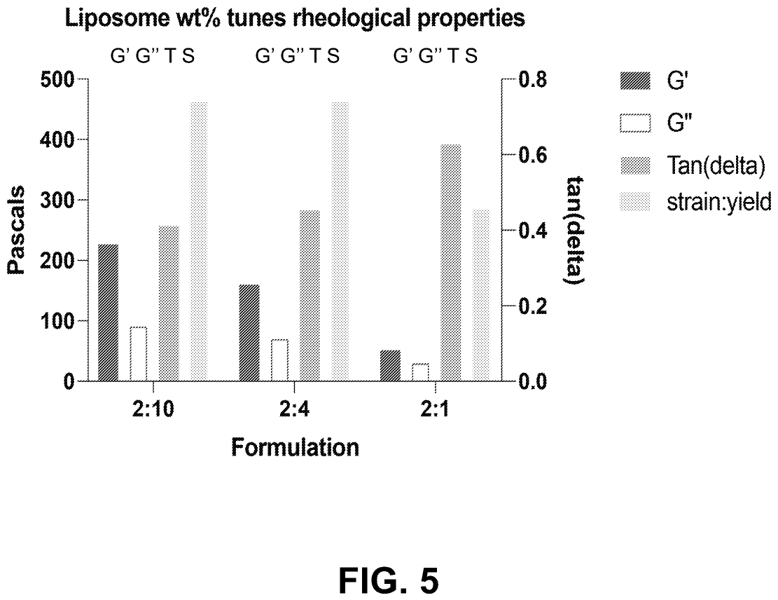

FIG. 5 shows a summary of key rheological parameters varying with liposomal content of hydrogels. G′, G″, and tan(delta) values taken for 10 rad/s.

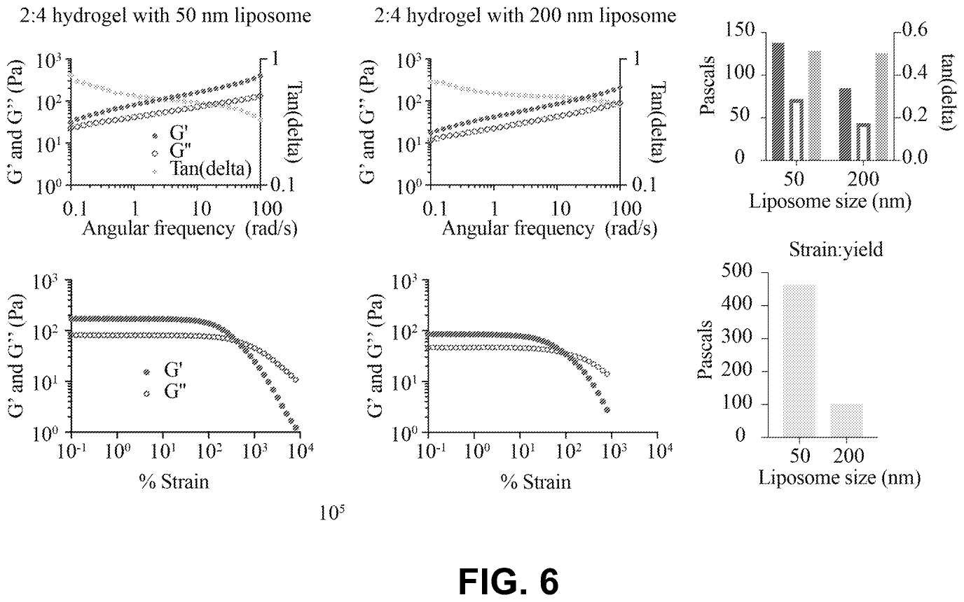

FIG. 6 shows dynamic rheological properties of polymer-liposome hydrogels are further tunable by altering the size of liposomes within the formulation.

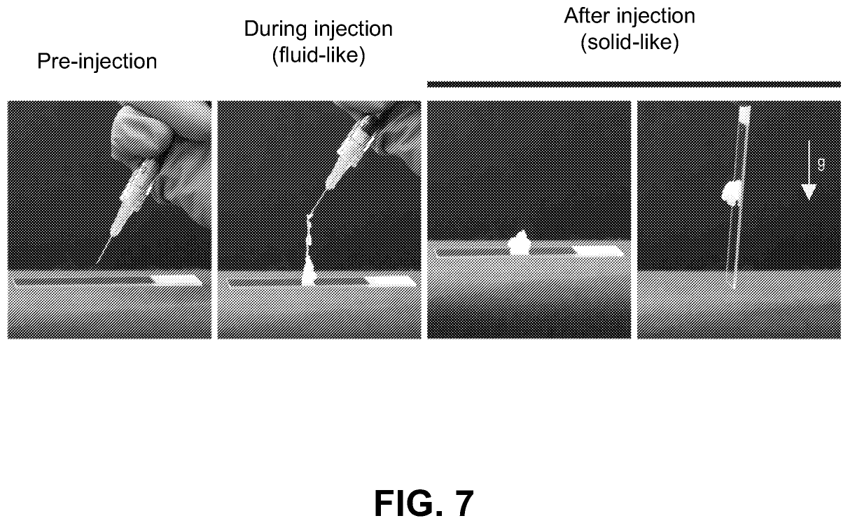

FIG. 7 shows polymer-liposome hydrogels are injectable through fine needles for minimally invasive clinical usage.

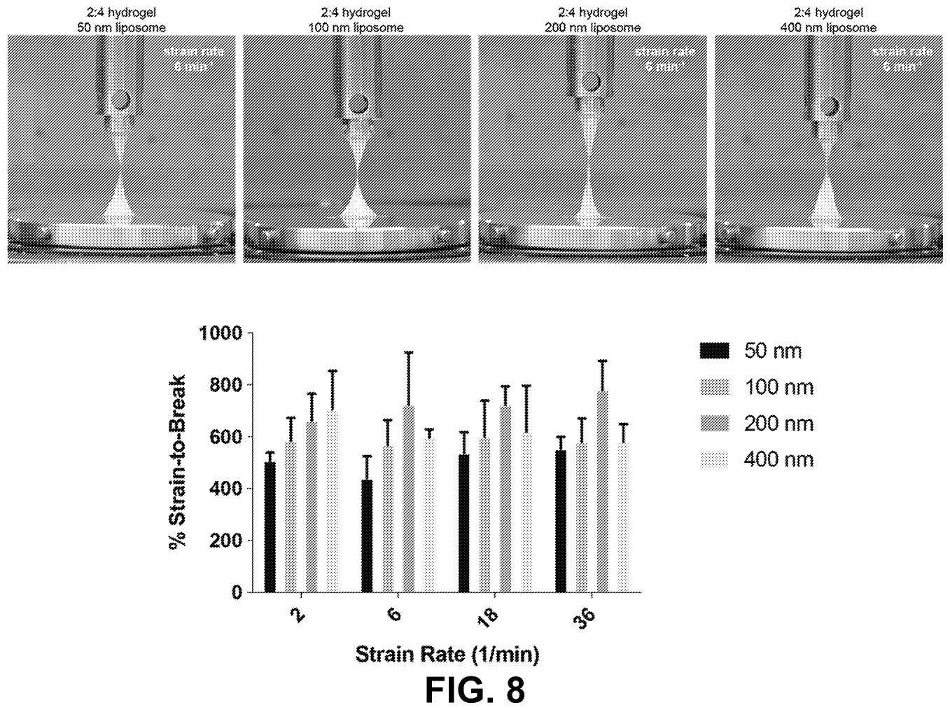

FIG. 8 shows polymer-liposome hydrogels possess extensional rheological attributes that are tunable by the size of the liposomes implemented in the formulation.

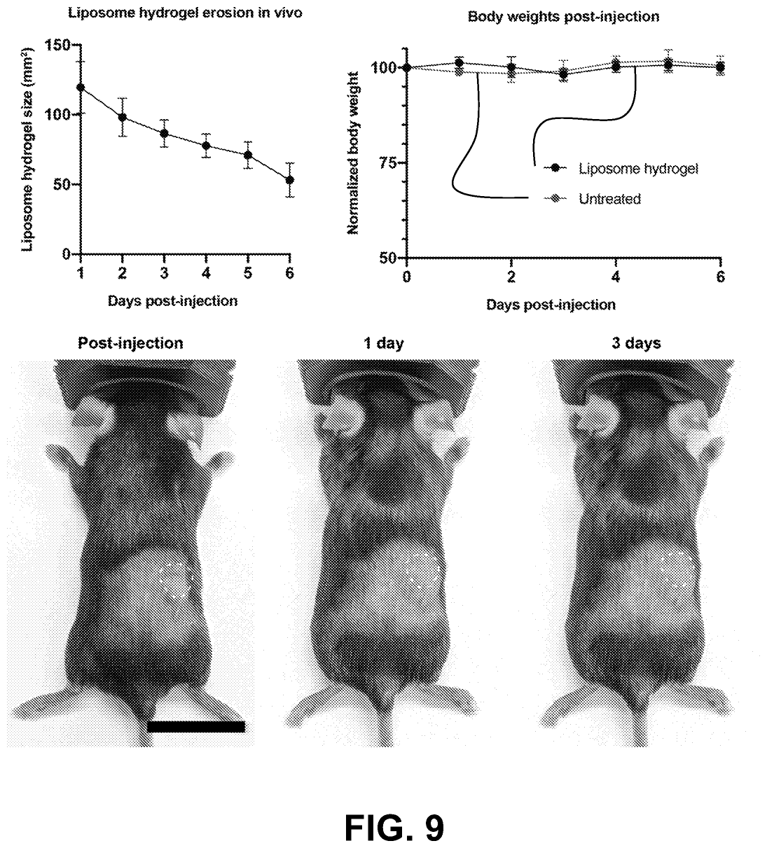

FIG. 9 shows polymer-liposomes are biocompatible. These hydrogels exhibit controlled erosion in vivo in C57BL/6 mice and do not provoke any signs of toxicity or inflammation. Scale bar is 25 mm.

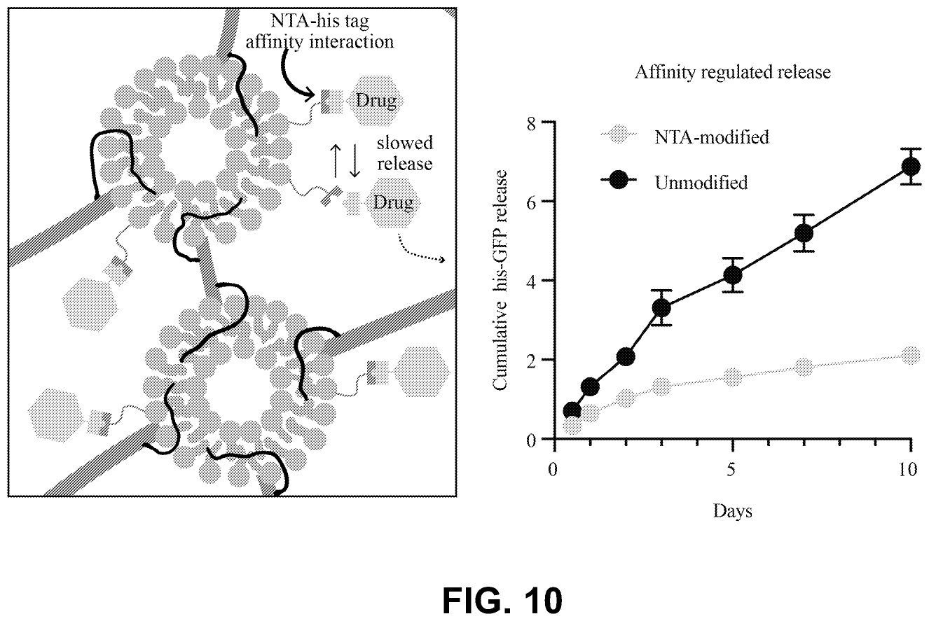

FIG. 10 shows surface chemistry of liposomes can be engineered to provide additional functionality to polymer-liposome hydrogels. Demonstrated is the inclusion of NTA-modified phospholipids into the liposomal component of the hydrogels. The resulting hydrogel is able to slow the release of a model his-tagged protein (GFP) in vitro. Data are % of total mass released based on amount loaded into hydrogels.

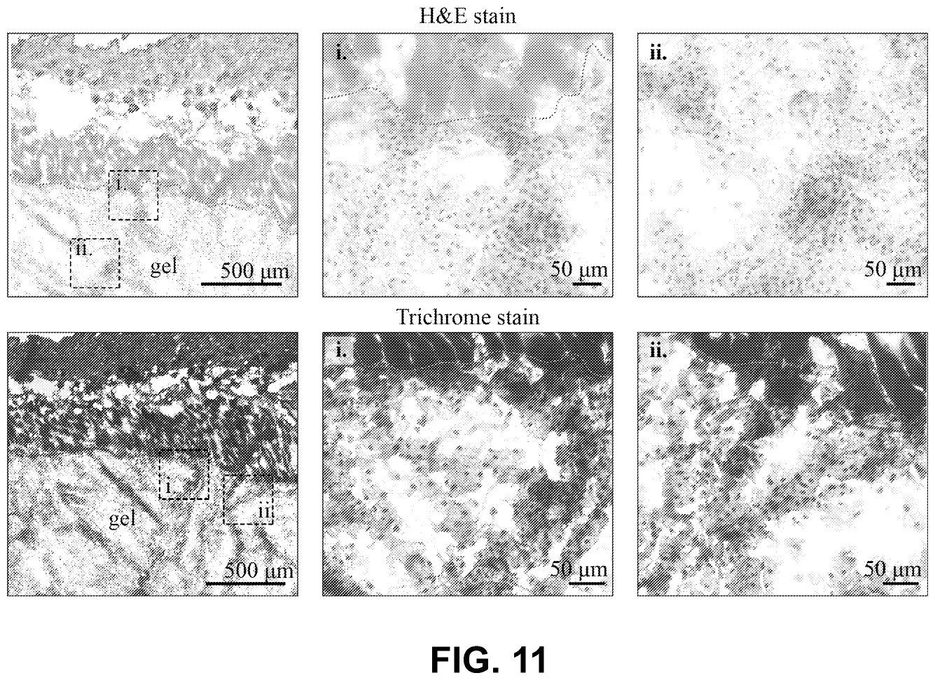

FIG. 11 shows histological analysis of explanted liposomal hydrogels using H&E or trichrome staining indicate minimal inflammatory response to the material following a week of implantation in immunocompetent mice. These indicate an overall lack of a foreign body response which indicates the material is nonimmunogenic and suitable for long term and sustained drug delivery applications.

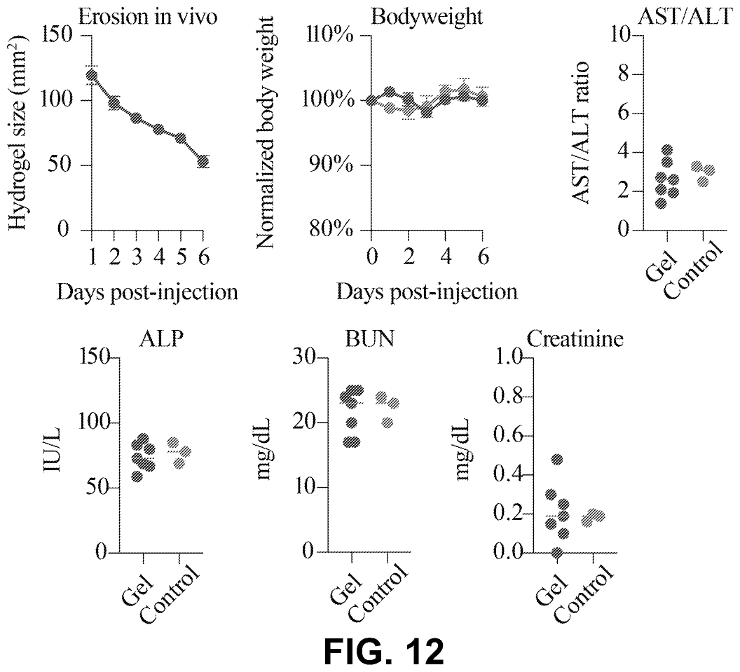

FIG. 12 shows further data demonstrating safety and biocompatibility in immunocompetent mice. Caliper measurements of hydrogel depot size over time indicates steady erosion of the material in vivo. Stable body weights of hydrogel treated mice are consistent with body weights of untreated mice. Blood chemistry panel on serum collected 7 days after hydrogel injection. Liver function was tracked by measuring AST/ALT ratio and ALP levels. Kidney function was tracked by measuring serum BUN and creatinine levels. All blood chemistry measures were consistent between hydrogel treated and untreated control mice.

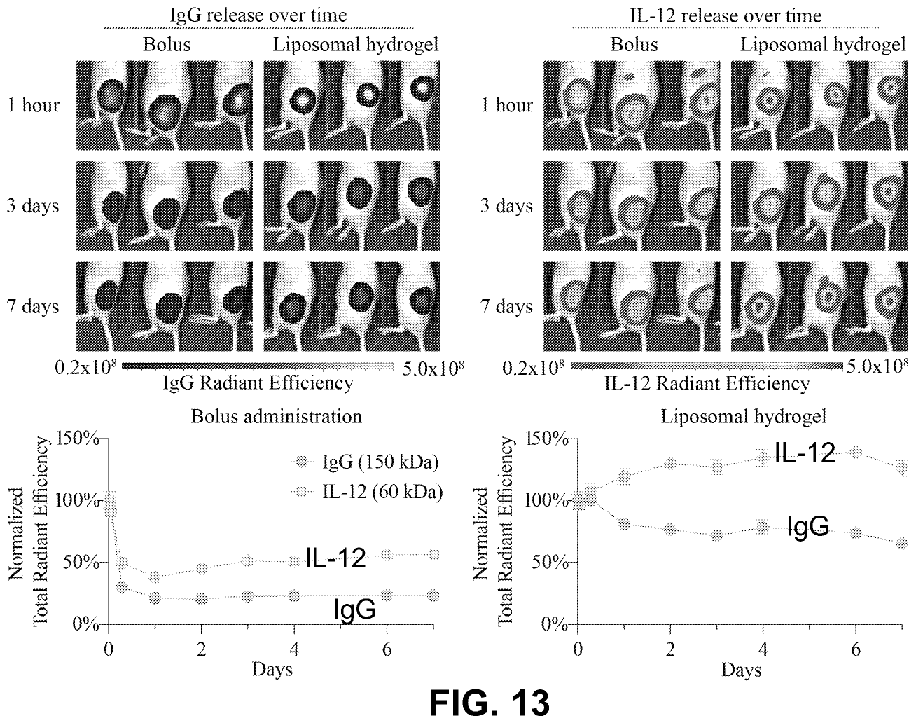

FIG. 13 shows liposomal hydrogels enable synchronized and sustained co-delivery of IgG antibody and IL-12 cytokine cargo in vivo, despite substantial size difference. Liposomal hydrogel mesh size is able to significantly slow release of bulky IgG antibodies, while anionic liposomes regulate release of small IL-12 cytokines via electrostatic interactions. Representative IVIS images of Alexa Fluor 594 labeled IgG and Alexa Fluor 700 labeled IL-12 release overtime. Quantified release curves of IgG and IL-12 following local subcutaneous bolus injection or subcutaneous injection of liposomal hydrogel.

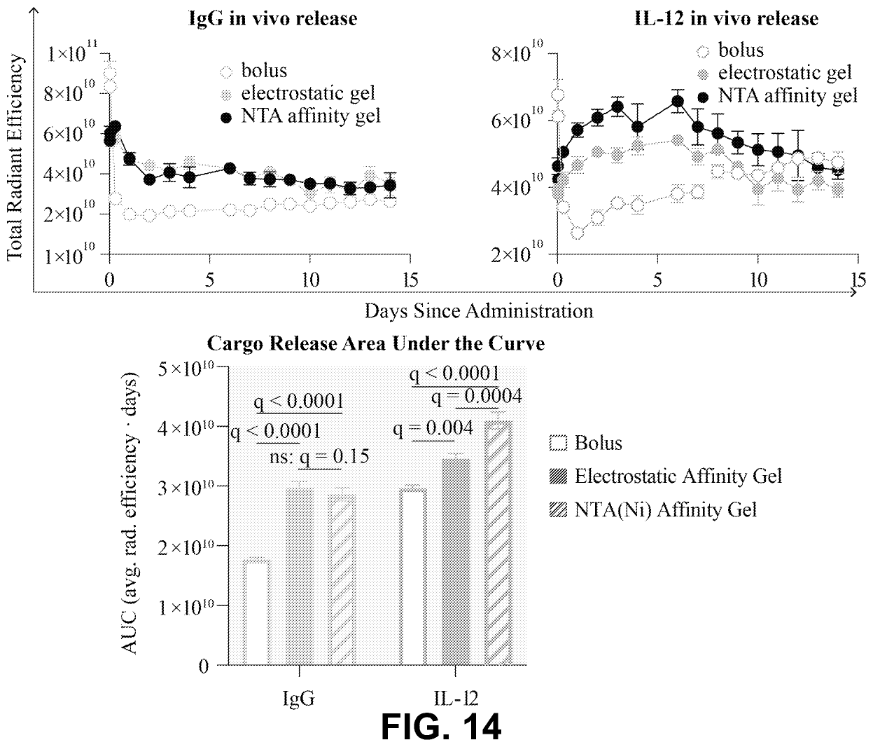

FIG. 14 shows IL-12 release in vivo is tuned by liposome surface chemistry, whereas IgG which lacks the affinity motif is unaffected by surface chemistry change. These data indicate this technology is capable of orthogonally modifying the release properties of co-encapsulated proteins. Mice were injected with liposomal hydrogels containing fluorescently labeled IgG and his-tagged IL-12. Area under the drug-time curve was calculated for in vivo release data for IgG and IL-12 for each administration method.

What is claimed is:

1. An injectable hydrogel network, comprising:
a non-covalently cross-linked hydrogel network with polymers functionalized with hydrophobic fatty pendant groups hydrophobically cross-linked with liposomal nanoparticles in which the liposomal nanoparticles are the cross-linkers.

2. The injectable hydrogel network as set forth in claim 1, wherein the polymers functionalized with hydrophobic fatty pendant groups are dodecyl-modified hydroxypropyl methyl cellulose.

3. The injectable hydrogel network as set forth in claim 1, wherein the liposomal nanoparticles further comprising affinity motifs capable of carrying and delivery of cargo.

4. The injectable hydrogel network as set forth in claim 1, wherein the liposomal nanoparticles further comprising phospholipids capable of carrying and delivery of cargo.

5. The injectable hydrogel network as set forth in claim 1, wherein the hydrophobic fatty pendant groups are defined as covalently attached functional groups bearing hydrophobic chemistries.

6. The injectable hydrogel network as set forth in claim 5, wherein the attached functional groups are alkyl, alkenyl, alkynl, or aryl functional groups.
